(19) United States
(12) Patent Application Publication
(10) Pub. No.: US 2009/0216305 A1
(43) Pub. Date: Aug. 27, 2009

(54) Thermal, outer layer leg wrap device

(76) Inventor: Larry Herman Bonner, Lawrenceburg, TN (US)

Correspondence Address: Michael G. Cameron, Esq., 2025 Savannah Drive, McKinney, TX, 75070, US

(21) Appl. No.: 12/378,562

(22) Filed: Feb. 17, 2009

Related U.S. Application Data
(60) Provisional application No. 61/066,807, filed on Feb. 22, 2008.

(51) Int. Cl. A61F 7/00; A41B 9/00

(52) U.S. Cl. 607/108; 2/109

(57) ABSTRACT

A thermal, outer layer leg wrap adapted for use by a handicapped, non-ambulatory or mobility restricted individual. The described embodiment of the present invention advantageously keeps the entire leg warm for sustained periods of time without having to remove the garment and interrupt the warmth. The described embodiment of the present invention comprises a separate wrap for each leg, thus increasing the ability of the individual wearing same to be more active and better able to be transferred while maintaining leg warmth.

### CROSS REFERENCE TO RELATED APPLICATION

This application claims the benefit of U.S. Provisional Application No. 61/066,807, filed on Feb. 22, 2008, entitled “THERMAL, OUTER LAYER LEG WRAP DEVICE.”

### TECHNICAL FIELD

The described embodiment of the present invention relates to thermal outer clothing adapted for use by a handicapped, non-ambulatory or mobility restricted individual.

### BACKGROUND

Thermal insulation materials are used to reduce the rate of heat transfer, for example, from a body to the surrounding atmosphere. The human body naturally radiates heat energy. The flow of heat from limbs of the human body can be retarded by covering them in thermal insulation, and the rate of heat transfer is dependent on the physical properties of the material employed to do this.

Damp materials may lose most of their insulating properties. The choice of insulation often depends on the means used to manage moisture and condensation on one side or the other of the thermal insulator. Clothing insulation depends on this aspect to function as expected. To combat cold, evacuating skin humidity is still essential while several layers may be necessary to simultaneously achieve this goal while matching one's internal heat production to heat losses due to wind, ambient temperature, and radiation of heat into space.

General clothing textiles are required by federal government regulations to meet burn rate standards found in 16 CFR 1610. This is a 45° burn test which sets criteria for how fast a flame can travel a specified distance. There are also similar federal standards for children's sleepwear in 16 CFR Section 1615 and 16 CFR Section 1616.

For a typical able bodied person with good blood circulation, conventional clothing serves its thermal insulation purposes well. However, for people who have, e.g., spinal cord injuries, circulation to the legs is decreased causing a persistent problem with what is known colloquially as “cold legs”. The problem of cold legs causes pain and discomfort and may interfere with lifestyle activities. A person suffering this affliction may restrict their activities to staying in bed or indoors in an attempt to keep their legs warm, yet this is often to no avail. Other ailments that result in cold legs include poor circulation, diabetes, peripheral arterial and vascular disease.

Conventional products available to these individuals do not wrap the entire leg therefore the back of the legs remain cold. A similar product is a leg warmer designed for able bodied persons, either as a fashion accessory or as a dance accessory. However, such products are typically made of a tight elastic fabric that must be pulled onto the leg which is not appropriate for patients who, e.g., have spinal cord injuries, or are otherwise handicapped, non-ambulatory or mobility restricted.

Also, some products enclose both legs in a “bag” which, disadvantageously, does not allow for transferring a patient, e.g., from a wheelchair to a bed, without having to remove the product. Many products used to address cold legs also, disadvantageously, get caught in the wheels of the wheelchair. What is desired is a thermal, outer layer leg wrap that overcomes the cited disadvantages.

### SUMMARY

The described embodiment of the present invention is a thermal, outer layer leg wrap adapted for use by a handicapped, non-ambulatory or mobility restricted individual. The described embodiment of the present invention advantageously keeps the entire leg warm for sustained periods of time without having to remove the garment and interrupt the warmth. The described embodiment of the present invention comprises a separate wrap for each leg, thus increasing the ability of the individual wearing same to be more active and better able to be transferred while maintaining leg warmth. The design of the described embodiment of the present invention is such that the upper end thereof terminates mid-thigh as to not interfere with the need for insertion and removal of medical devices, such as catheters.

### BRIEF DESCRIPTION OF THE FIGURES

The invention will be better understood and its numerous objects and advantages will become more apparent to those skilled in the art by reference to the following figures, in conjunction with the accompanying specification, in which:

FIG. 1 is a front view of a pair of an embodiment of the leg wraps of the present invention as worn by a user;

FIG. 2 is an exploded view of a leg wrap of an embodiment of the present invention;

FIG. 3 is a front view of an embodiment of the present invention as worn by a user including handwarmer and cargo pockets;

FIG. 4 is a view of the substantially all of the outside layer of a described embodiment of the present invention; and

FIG. 5 is a view of the described embodiment of the present invention as worn by a user.

### DETAILED DESCRIPTION

The described embodiment of the present invention is a thermal, outer layer leg wrap adapted for use by a handicapped, non-ambulatory or mobility restricted individual. The described embodiment of the present invention advantageously keeps the entire leg warm for sustained periods of time without having to remove the garment and interrupt the warmth. The described embodiment of the present invention comprises a separate wrap for each leg, thus increasing the ability of the individual wearing same to be more active and better able to be transferred while maintaining leg warmth. The design of the described embodiment of the present invention is such that the upper end thereof terminates mid-thigh as to not interfere with the need for insertion and removal of medical devices, such as catheters.

The described embodiment of the present invention achieves several objectives: to keep all sides of each leg warm; to maintain such thermal, outer layer leg wrap in place when the wearer is active; to maintain the thermal, outer layer leg wrap in place while transferring in and out of a bed or a vehicle; to provide a thermal, outer layer leg wrap that does not interfere with the function of a wheelchair; and to provide a thermal, outer layer leg wrap that does not interfere with the medical care of the wearer.

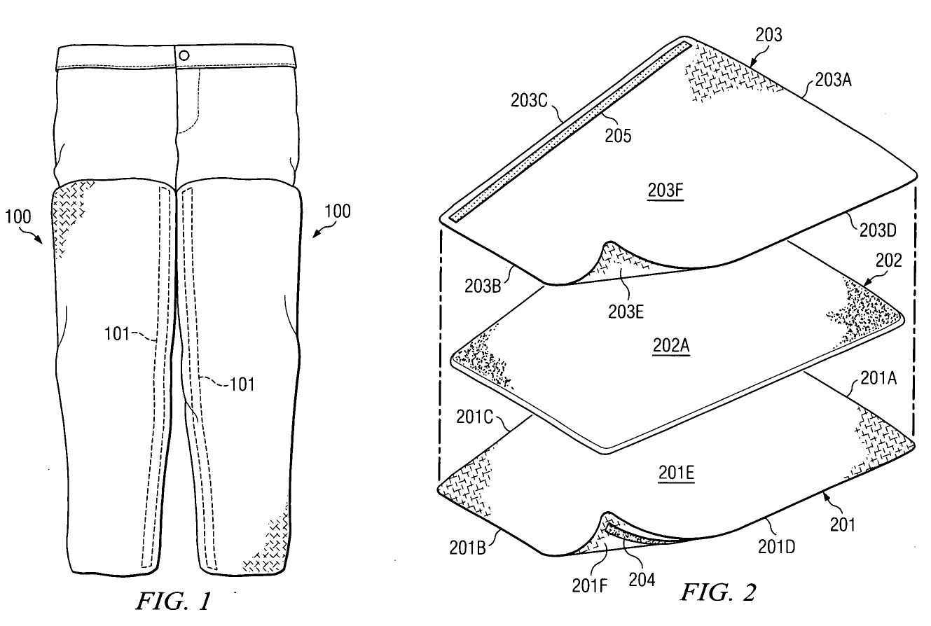

FIG. 1 is a front view of an embodiment of a pair of the leg wraps 100 having a coupling means 101, such as a loop and hook (e.g., Velcro) attachment or zipper or other such means, running longitudinally along the sides of the leg wrap 100.

Referring now to FIG. 2, a exploded view of the leg wrap 100 illustrates the physical structure of the leg wrap 100. As seen therein, the leg wrap 100 has three layers of material sewn together, an inner layer 201, a middle, insulating layer 202 and an outer layer 203. More specifically, the inner layer 201 has a top edge 201 A, bottom edge 201B, left edge 201C and right edge 201D, inner surface 201E and outer surface 201F and having a generally tapered shape (the narrowest portion of the taper being at the bottom edge 201 B); outer layer 203 having a top edge 203A, bottom edge 203B, left edge 203C and right edge 203D, inner surface 203E and outer surface 203F having a generally tapered shape corresponding to the generally tapered shape of the inner layer 201; a middle, insulating layer 202 having material 202A layered between the inner surface 201E of the inner layer 201 and the inner surface 203E of the outer layer 203; the outer edges 201A, 201B, 201C and 201D of the inner layer 201 being coupled to the outer edges 203A, 203B, 203C and 203D of the outer layer 203; and the outer surface 201F of the inner layer 201 having a first coupling means 204 arranged longitudinally and adapted to couple to a second coupling means 205 arranged longitudinally on the outer surface 203F of the outer layer 203, the leg wrap 100 being adapted to be wrapped around a leg and being coupled together by the first coupling means 204 and second coupling means 205. Each of the first and second coupling means 204, 205 can comprise, e.g., a loop and hook (e.g., Velcro®) attachment or zipper or other such means, running longitudinally along the outer surface 201F of the inner side 201 and outer surface 203F of the outer layer 203 of the leg wrap 100.

Each of the inner, middle, insulating and outer layer is comprised of materials depending on its desired functionality. For example, the inner layer 201 can be a material adapted to manage moisture and condensation. The outer layer 203 can be made of a quilted material. Either or both of the inner layer 201 and outer layer 203 can be comprised of a poly fleece material or a blended cotton-polyester material. The middle insulating layer 202 is preferably comprised of, e.g., a batting or wadding material made of cotton, polyester, wool, waterfowl down, synthetic fiberfill or a blend thereof. Such material would be chosen based on the environment in which it is anticipated that the described embodiment of the present invention would be used. For example, a specific embodiment of the present invention may be designed to allow it to breathe, allowing moisture to pass through and evaporate. Other objectives may include resiliency; the middle layer 202 being adapted to be compressed to a small area, spring back and retains loft; conform to leg shape without undesirable bulk; be machine washable and dryable; be lightweight to provide warmth without weight and excess bulk; and be clean, odorless and non-allergenic.

Several companies manufacture polyester fiberfill for use in insulated garments. Hollofil comprises a series of short, hollow fibers meshed together. The short fiber is more down-like in terms of loft, while the hollow structure reduces the weight. Hollofil is heavier than down but lighter than other polyester fiberfills. Hollofil is less stable than Needlepunch fiberfill batts and, like down, requires more quilting lines and compartments to keep the fiber from shifting in the garment. Quallofil is designed to most closely duplicate down. It features four internal channels to increase thermal performance. Quallofil is known for its resiliency, softness, loft, and ability to maintain 90% of its insulation qualities when completely saturated with water. Thinsulate, Thermolite, Texolite Plus and Thermal R are examples of fiberfill products that provide warmth without the bulk of down or conventional polyester fiberfills. Thinsulate, 65% olefin/35% polyester, and Thermolite, 100% polyester, feature tiny micro fibers blown to form a high density web. The extremely fine fibers provide an increased surface area to efficiently trap air for insulation. Thermolite features an additional sheath that surrounds the micro fiber core. Texolite Plus and Thermal R feature metallized films combined with either polyester fiberfill or foam. The films serve as reflective barriers to conserve energy. In addition, Thermal R offers windproof, breathable and non-allergenic properties. Needlepunch is a type of polyester insulation material. The web structure is formed by interlacing many polyester fibers by punching with barbed needles. This creates a felt-like fabric that is warm, lightweight and inexpensive. This more compact material is often used in outerwear, with a minimum of quilting lines. Polyester fibers used in needlepunch construction are non-allergenic, machine washable and dry cleanable.

Further, the middle layer 202 may have contained therein electrically powered heating pads. Such heating pads may be formed from wire that is insulated or padded. Alternatively, such middle layer 202 may have conductive or heating properties. For example, the middle layer 202 may consist of, among other things, an electrically conductive fiber that is fabricated by adding copper sulfide in a fiber; or a fabric having an electrically conductive polymer layer thereon; or a combination of a synthetic fiber fabric, polyurethane foam, polyethylene film, flexible circuit of aluminum foil, and cotton cloth, wherein the aluminum foil serves as the heating element. Finally, the middle layer 202 may comprise a laminar resistive heating element with a polymer having a conductive filler, such as graphite or carbon black.

Different fabrics can be used on the outer layer 203 and the inner layer 201. In an embodiment of the present invention, a tightly woven water- and wind-resistant fabric is used for the outer layer 203. Water repellent properties are less important, but wind resistance helps retain body heat and warmth. The chosen fabric may also be chosen based on its high tear strength. The embodiment of the present invention that uses down filling or insulation should have downproof outer fabrics. One such outer layer 203 can be nylon taffeta, a tightly woven fabric with a smooth finish. The closely packed yarns provide good wind resistance, abrasion resistance and water repellency, although a finish for the latter is often added. Nylon taffeta is breathable, downproof and machine washable. If the described embodiment of the present invention uses this fabric, the seam edges should be treated or seared with a flame to avoid yarn slippage and raveling. Other properties include snag resistance, high tear strength and heat sensitivity. Ripstop nylon is similar to nylon taffeta, although usually lighter in weight. The unique feature is a heavy nylon thread at ¼″ intervals running both lengthwise and crosswise in the fabric. Poplin is a cotton or polyester/cotton blend woven cloth. When tightly woven the fabric provides good abrasion and wind resistance, although a water-repellent finish must be applied. Versatech is a 100% polyester woven fabric that is breathable and water repellent. The super fine polyester yarn is woven into a very dense and drapable fabric. It prevents water droplets from penetrating the weave, yet water vapor passes between the yarns for breathability and comfort. Possessing the same comfort and protection properties is Ultrex®. This 100% nylon fabric features layers of a microporous coating and a water repellent finish. Many fabrics used in outdoor wear feature a Gore-Tex® coating. This microporous film, when applied to conventional fabrics such as cotton, polyester/cotton poplin, or nylon taffeta creates a waterproof barrier. Tiny pores in the film prevent water droplets from penetrating, yet allow moisture vapors from body heat and perspiration to escape. Fabrics treated with Gore-Tex® or other microporous films, including Bion II® and Dicrylan®, will be more costly than similar untreated fabrics, but provide improved protection and comfort. Versatech®, Ultrex®, and Gore-Tex® are three of an increasing number of fabrics with similar properties. Fashion fabrics such as corduroy and suede cloth may also be used, although water repellent finishes must be applied. Some fabric colors and finishes are designed to reflect light and can be selected when the safety of the patient is a major consideration. The use of elastic or belted top and bottom edges are used to prevent cold air from traveling up under the described embodiment of the present invention.

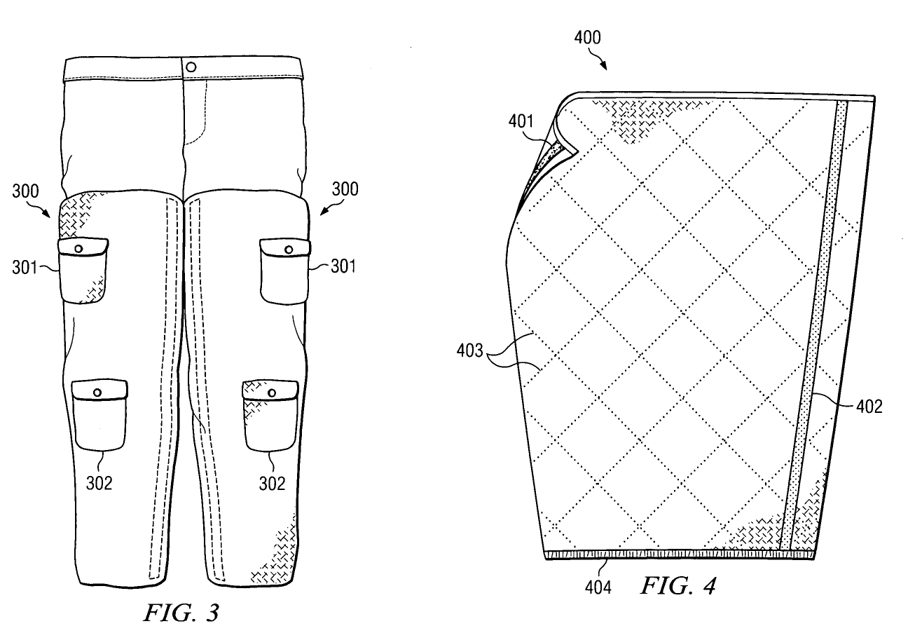

As seen in FIG. 3, an embodiment of the present invention can further include handwarmer pockets 301 with additional insulation material provide comfort and protection. Further, cargo pockets 302 can be added to the thermal, outer layer leg wraps 300 with button flap or zipper secure keys, identification or other valuables.

Quilting stitches are decorative but also make important functional contributions to the described embodiment of the present invention. The design of quilting stitches affects the distribution of down and synthetic fiberfills throughout the life of the leg wrap. Without quilting stitches, down will shift and polyester fiberfill will either shift or pull apart. Quilting stitches placed lengthwise or vertically preferably are to be avoided. During wear, insulation material, especially down, will shift and slip to the bottom edge of the leg wrap, reducing insulation. Crosswise quilting lines which go around the legs are the preferred stitching design of the described embodiment of the present invention. Insulation is held in place and more evenly distributed over the leg. The chevron stitch design results in shorter compartments and holds down and fiberfill in place.

FIG. 4 provides a view of the substantially all of the outside layer of a described embodiment of leg wrap 400 of the present invention showing loop and hook strips 401, 402 with the middle fill layer held in place with crosswise quilting stitches 403 stitched through the inner layer, middle layer and outer layer. As seen therein, the longitudinal length of the leg wrap of the described embodiment of the present invention could be tailored for the wearer, and would generally equal the length of the wearer's ankle to mid-thigh (approximately 18 inches to 32 inches). In a further embodiment of the present invention, a light elastic strip 404 is included at the bottom edge corresponding to where the thermal, outer layer leg wrap would meet the wearer's ankle. In a further embodiment, an upper end of each leg wrap terminates mid-thigh as to not interfere with the need for insertion and removal of medical devices, such as catheters.

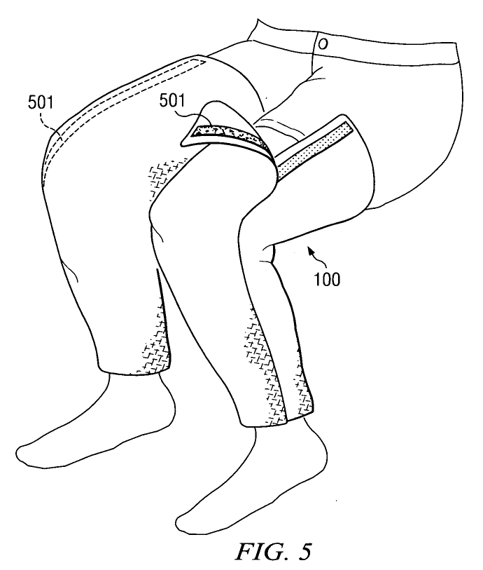

In operation, the described embodiment of the present invention is adapted to open completely and lay flat. This allows the patient to lay the garment down under the legs and then wrap the garment 100 around the legs to be worn as seen in FIG. 5. In the embodiment of the present invention which uses a Velcro® attachment 501 along the longitudinal edges of the leg wrap, the hook portion of the Velcro® along one longitudinal edge is adapted to attach to the loop portion of the Velcro along the other longitudinal edge. A single thermal, outer layer leg wrap is used for each leg.

Because the described embodiment of the present invention wraps each leg closely it advantageously keeps each leg warmer than other products or a blanket on the legs. The described embodiment of the present invention can easily be placed on the wearer as it does not require fine motor skills to enclose the leg wrap around the leg. Hence, the patient having limited motor skills, as well as the health care professional, can easily fit the thermal, outer layer leg wrap on the patient's legs.

In a further embodiment of the present invention, elastic can be added at the top edge allowing an additional population of greater mobility to use the product. Different embodiments of the present invention based on size and colors of material allow the leg warmers to be adapted to any of men, women or children. A further embodiment of the present invention includes a pocket sewn inside the top of the thermal, outer layer leg wrap.

The embodiments shown and described above are only exemplary. Even though numerous characteristics and advantages of the described embodiment of the present invention have been set forth in the foregoing description together with details of the invention, the disclosure is illustrative only and changes may be made within the principles of the invention to the full extent indicated by the broad general meaning of the terms used in the attached claims.

What is claimed is:

1. An item of thermal clothing, comprising:
an inner layer having a top edge, bottom edge, left edge and right edge and inner surface and outer surface and having a generally tapered shape;
an outer layer having a top edge, bottom edge, left edge and right edge and inner surface and outer surface having a generally tapered shape corresponding to the generally tapered shape of the inner layer;
a middle, insulating layer of material between the inner surface of the inner layer and the inner surface of the outer layer;
the top edge, bottom edge, left edge and right edge of the inner layer being coupled, respectively, to the top edge, bottom edge, right edge and left edge of the outer layer; and
the outer surface of the inner layer having a first coupling means arranged longitudinally and adapted to couple to a second coupling means arranged longitudinally on the outer surface of the outer layer, the item of thermal clothing adapted to be wrapped around a leg and coupled by the first coupling means and second coupling means.

2. The item of thermal clothing of claim 1, wherein the middle, insulating layer is made of a batting material.

3. The item of thermal clothing of claim 1, wherein the inner layer and outer layer are made of a flame resistant material.

4. The item of thermal clothing of claim 1, further having a heating means within the middle layer adapted to receive energy from an external power source and radiate heat.

5. The item of thermal clothing of claim 1, wherein one or both of the inner layer and outer layer are comprised of poly fleece material.

6. The item of thermal clothing of claim 1, wherein one or both of the inner layer and outer layer are comprised of blended cotton-polyester material.

7. The item of thermal clothing of claim 2, wherein the upper end thereof terminates mid-thigh as to not interfere with the need for insertion and removal of medical devices, such as catheters.

8. The item of thermal clothing of claim 1, further comprising handwarmer pockets located on the outer layer.

9. The item of thermal clothing of claim 1, further comprising cargo pockets located on the outer layer.

10. The item of thermal clothing of claim 9, wherein the cargo pockets include a button flap closure.

11. The item of thermal clothing of claim 9, wherein the cargo pockets include a zipper closure means.

12. The item of thermal clothing of claim 1, wherein the middle insulating layer comprises a down material held in place with crosswise quilting stitches stitched through the inner layer, middle layer and outer layer.

13. The item of thermal clothing of claim 1, wherein the middle insulating layer comprises polyester fiberfill held in place with crosswise quilting stitches stitched through the inner layer, middle layer and outer layer.

14. A leg wrap device, comprising:
an inner layer having a top edge, bottom edge, left edge and right edge and inner surface and outer surface and having a generally tapered shape;
an outer layer having a top edge, bottom edge, left edge and right edge and inner surface and outer surface having a generally tapered shape corresponding to the generally tapered shape of the inner layer;
a middle, insulating layer of material between the inner surface of the inner layer and the inner surface of the outer layer;
the top edge, bottom edge, left edge and right edge of the inner layer being coupled, respectively, to the top edge, bottom edge, right edge and left edge of the outer layer; and
the outer surface of the inner layer having a first coupling means arranged longitudinally and adapted to couple to a second coupling means arranged longitudinally on the outer surface of the outer layer, the item of thermal clothing adapted to be wrapped around a leg and coupled by the first coupling means and second coupling means.

15. The leg wrap of claim 14, further comprising cargo pockets located on the outer layer.

16. The leg wrap of claim 14, wherein the cargo pockets include a button flap closure.

17. The leg wrap of claim 14, wherein the cargo pockets include a zipper closure means.

18. The leg wrap of claim 14, wherein the middle insulating layer comprises wherein the middle insulating layer comprises a down material held in place with crosswise quilting stitches stitched through the inner layer, middle layer and outer layer.

19. The leg wrap of claim 14, wherein the middle insulating layer comprises wherein the middle insulating layer comprises polyester fiberfill held in place with crosswise quilting stitches stitched through the inner layer, middle layer and outer layer.

20. The leg wrap of claim 14, wherein the outer layer is a nylon taffeta.
